(19) United States
(12) Patent Application Publication
(10) Pub. No.: US 2022/0288416 A1
(43) Pub. Date: Sep. 15, 2022

(54) HAIR GROWTH STIMULATING SYSTEM

(71) Applicant: Leslie Owen Paull, Austin, TX (US)

(72) Inventor: Leslie Owen Paull, Austin, TX (US)

(21) Appl. No.: 17/202,282

(22) Filed: Mar. 15, 2021

(51) Int. Cl. A61N 5/06; A61K 9/00; A61K 31/425; A61K 38/06; A61K 9/10; A61K 9/51

(52) U.S. Cl. CPC A61N 5/0617; A61K 9/0014; A61K 31/425; A61K 38/063; A61K 9/10; A61K 9/5115; A61N 2005/063; A61N 2005/0647; A61N 2005/0663; B82Y 5/00

(57) ABSTRACT

The present invention discloses a hair growth stimulating system. The hair growth stimulating system includes a topical solution for application on a user's scalp with nanoparticles dispersed throughout. The nanoparticles emit red light toward the scalp to stimulate hair growth when irradiated with photons from a light source. The present invention also discloses a clothing article for hair growth stimulation that includes a head covering for overlaying a scalp of a user. The head covering has a window to allow light to pass through to the scalp. The window may have a thin film embedded with nanoparticles that emit red light toward the scalp to stimulate hair growth when irradiated with photons from a light source. Alternatively, the window has a thin film for receiving a topical solution that includes nanoparticles dispersed therein, which in turn emit red light toward the scalp to stimulate hair growth when irradiated with photons.

### TECHNICAL FIELD

The present invention relates to the field of hair growth stimulating systems, including clothing articles for hair growth stimulation.

### BACKGROUND ART

Hair loss is a condition affecting a substantial percentage of the population. In fact, pattern hair loss by age fifty (50) affects about half of men and a quarter of women. Hair loss commonly presents as androgenetic alopecia, or pattern hair loss. In men, hair loss usually begins above the temples, and the receding hairline eventually forms a characteristic “M” shape; hair at the top of the head also thins, often progressing to baldness. In women, androgenetic alopecia begins with gradual thinning at the part line, followed by increasing diffuse hair loss radiating from the top of the head. While most hair loss results from androgenetic alopecia, about two (2) percent of people develop alopecia areata at some point in time.

Regardless of the type of hair loss, individuals affected by this condition are generally self-conscious about their hair loss. These individuals often perceive themselves as less attractive and/or older looking. For these reasons, a variety of attempts have been made to improve an individual's appearance by restoring the appearance of a full head of hair.

First attempts to address hair loss were mechanical in nature and focused on the use of hair pieces, such as toupees or wigs, to be fixed topically over the area of the scalp missing or losing natural hair. The problems with such hair pieces included that it was difficult to simulate an individual's natural hair color and texture, resulting in a fake look. In addition, such hair pieces were difficult to secure in place during physical activity or when wearing a hat, scarf, or other accessory. Further, body oils had a tendency to build up below hair pieces worn for any period of time, causing bacteria to grow on the skin underneath and the generation of an unpleasant odor.

More complicated mechanical solutions such as “hair weaving” were also developed. Hair weaving involved the addition of human or synthetic hair to bald areas of scalp or to a personal's natural hair, and could be performed by non-medical personnel or other service providers in the cosmetic field. Hair weaving, like hair pieces, had certain advantages, such as being a relatively fast solution to the problem of hair loss. However, weaving also shared many disadvantages with hair pieces, including a fake appearance and the development of bacteria. In addition, hair weaves required routine maintenance, and would often become brittle and dry, as the hair “woven” onto the scalp or natural hair did not receive nutrients from the scalp. Hair weaving was also often costly and could require several hours to complete.

Other attempts to address the problem of hair loss have been to surgically replace missing hair with “hair transplants,” formerly known as “hair plugs.” During a hair transplant, healthy hair follicles are harvested from one area of the scalp and transplanted to fill bald or balding areas. While hair transplant procedures have a high rate of success at keeping areas of the scalp previously impacted by hair loss covered with natural hair, transplants are complex and expensive. The drawbacks to these procedures include the fact that they require highly trained personnel and physicians to perform and also require patients to undergo a physical recovery period that can last for several weeks. During the recovery period, a patient may experience itchiness and must avoid direct sunlight to the scalp and sleep with the head and neck elevated.

Pharmaceutical products have also been developed to facilitate hair growth. However, these products are typically more effective at stopping hair loss than regenerating new hair. Further, such medication is expensive and is typically not covered by insurance. Patients must take the medication continuously, or risk losing any regrown hair. In addition, such medication may be dangerous for individuals with certain health conditions, such as heart problems. Side effects to pharmaceutical treatments can include skin irritation, dandruff, and an itchy scalp, as well as facial hair growth on women.

Finally, for over 20 years, lasers have also been used to stimulate hair growth. Laser hair therapy, however, requires multiple treatments to maintain any results, and treatment must be continued over an individual's lifetime. Treatments are costly and time-consuming, and may interact adversely with some medications. In addition, laser treatments require a trained operator, and prolonged exposure to lasers may burn the scalp and damage the skin. Further, while a technician is attempting to target certain areas of hair loss, an individual's natural hair may block the laser light, further reducing the potential effectiveness of the treatment.

### SUMMARY OF INVENTION

The present invention discloses hair growth stimulating systems. Hair growth stimulating systems according to embodiments of the present invention may include a topical solution for application on a scalp of a user and a plurality of nanoparticles dispersed in the topical solution. Such nanoparticles may emit red light toward the scalp to stimulate hair growth when irradiated with photons from a light source. Hair growth stimulating systems according to embodiments of the present invention may also include head coverings for overlaying a scalp of a user. The head covering may have a window to allow photons to pass through to the scalp. Such a window may in turn have a thin film embedded with nanoparticles emitting red light toward the scalp to stimulate hair growth when irradiated with the photons. Hair growth stimulating systems according to embodiments of the present invention may also include clothing articles for hair growth stimulation. The clothing article may include a head covering for overlaying a scalp of a user, the head covering having a window to allow light to pass through to the scalp. Such a window may have a thin film for receiving a topical solution that includes nanoparticles dispersed in the topical solution that emitting red light toward the scalp to stimulate hair growth when irradiated with the photons from a light source.

### BRIEF DESCRIPTION OF DRAWINGS

The accompanying drawings, which are incorporated in and constitute a part of this specification, illustrate an implementation of apparatus and methods consistent with the present invention and, together with the detailed description, serve to explain advantages and principles consistent with the invention. In the drawings,

FIG. 1 is a drawing illustrating a perspective view of an exemplary hair growth stimulating system according to embodiments of the present invention.

FIG. 1A is a drawing illustrating a cut-away view of an exemplary container of topical solution useful in the exemplary hair growth stimulating system of FIG. 1 according to embodiments of the present invention.

FIG. 1B is a drawing illustrating a closeup view of an applied exemplary topical solution useful in the exemplary hair growth stimulating system of FIG. 1 according to embodiments of the present invention.

FIG. 2 is a drawing illustrating a perspective view of an exemplary hair growth stimulating system according to embodiments of the present invention.

FIG. 3 is a drawing illustrating a perspective view of an exemplary hair growth stimulating system according to embodiments of the present invention.

FIG. 4 is a drawing illustrating a perspective view of an exemplary clothing article for hair growth stimulation according to embodiments of the present invention.

FIG. 5A is a drawing illustrating a right view of the exemplary clothing article of FIG. 4 for hair growth stimulation according to embodiments of the present invention.

FIG. 5B is a drawing illustrating a cross-sectional view along the line A-A of an exemplary clothing article of FIG. 5 for hair growth stimulation according to embodiments of the present invention.

FIG. 5C is a drawing illustrating a bottom view of the exemplary clothing article of FIG. 4 for hair growth stimulation according to embodiments of the present invention.

FIG. 5D is a drawing illustrating a cross-sectional view of an exemplary clothing article similar to FIG. 4 for hair growth stimulation according to embodiments of the present invention along a line similar to A-A in FIG. 5A.

FIG. 6A is a drawing illustrating a chart of light wavelengths of incoming photons and outgoing photons of exemplary nanoparticles useful in exemplary clothing articles for hair growth stimulation according to embodiments of the present invention.

FIG. 6B is a drawing illustrating a chart of light wavelengths of incoming photons and outgoing photons of exemplary nanoparticles useful in exemplary clothing articles for hair growth stimulation according to embodiments of the present invention.

### DESCRIPTION OF EMBODIMENTS

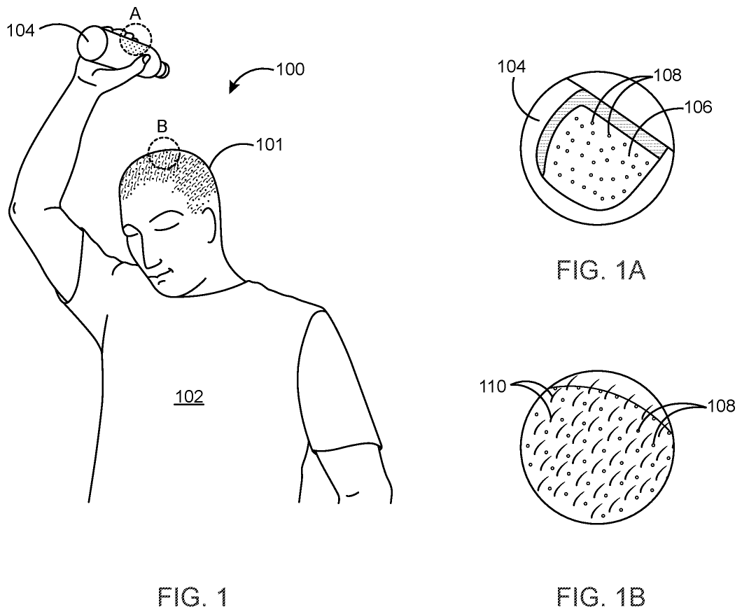

Exemplary embodiments of exemplary hair growth stimulating systems and exemplary clothing articles for hair growth stimulation are described herein with reference to the accompanying drawings, beginning with FIG. 1. FIG. 1 sets forth a drawing illustrating a perspective view of an exemplary hair growth stimulating system (100) according to embodiments of the present invention. In FIG. 1, the user (102) applies the exemplary hair growth stimulating system (100) by dispensing the system (100) from a container (104). The user (102) directs the hair growth stimulating system (100) to the scalp (101).

The exemplary hair growth stimulating system (100) of FIG. 1 is stored and transported in a container (104). The container may be a tube, made of plastic, rubber, or another flexible material, to permit squeezing. The container may also be made of other pliable materials, for example, or heavy cardboard. When an exemplary container is made from a material capable of being saturated by the exemplary hair growth stimulating system of FIG. 1, such a container may also include an interior liner to prevent saturation and the resulting failure of the container due to leakages from cracks or seeping. While in some embodiments, an exemplary container may be pliable or squeezable, in other embodiments a container may be rigid in structure. In such example, a user would pour or pump the exemplary hair growth stimulating system (100) from the container (104) of FIG. 1. The container may also be opaque, translucent, or clear material depending on a variety of factors such as marketing preferences, visual appeal of a particular hair growth stimulating system, or labeling preferences.

The container (104) of FIG. 1 has an outlet for dispensing the topical solution (104) on the scalp (101) of the user (102). The outlet may be an aperture that allows the topical solution (104) to be squeezed or poured out of the container (104). The outlet may also be an aperture in the form of a spray nozzle. In other embodiments, to remove the topical solution (106) from the container, the user (102) may push out the topical solution (106) from the bottom, or may scoop out the topical solution (106) using an instrument such as a spoon or a small trowel. The user (102) may also remove the topical solution (106) from the container with a suction or pumping mechanism.

The exemplary hair growth stimulating system (100) of FIG. 1 includes a topical solution (106) for application on a scalp (101) of a user (102). The topical solution (106) of FIG. 1 may be implemented as a cream, lotion, gel, or other liquid solution. The topical solution (106) may be oil based or water based. In some embodiments, the topical solution (106) of FIG. 1 may include alpha lipoic acid and/or glutathione, both of which are known to slow the rate of hair loss and or stimulate hair regrowth. The user (102) may apply the topical solution (106) of FIG. 1 to the scalp (101) in certain embodiments using their hand, a brush, a comb, a cloth, or a sponge.

For further explanation, FIG. 1A is a drawing illustrating a cut-away view of an exemplary container (104) of topical solution (106) useful in the exemplary hair growth stimulating system (100) of FIG. 1 according to embodiments of the present invention. FIG. 1A corresponds to region ‘A’ shown on FIG. 1. FIG. 1B is a drawing illustrating a closeup view of an applied exemplary topical solution (106) useful in the exemplary hair growth stimulating system (100) of FIG. 1 according to embodiments of the present invention. FIG. 1B corresponds to region ‘B’ shown on FIG. 1.

The exemplary hair growth stimulating system (100) of FIG. 1 includes nanoparticles (108) dispersed in the topical solution (106). The nanoparticles (108) of FIG. 1 may also be referred to as quantum dots. In certain embodiments, quantum dots are semiconductor particles a few nanometers in size containing at least one electron. When quantum dots are illuminated by certain light sources, an electron in the quantum dot can be excited to a state of higher energy. When the excited electron drops back into a lower state of energy, the quantum dot releases that change in energy by emitting light. The color of that light depends on the energy difference between the energy bands of the electrons for that particular quantum dot. Generally, the larger sized quantum dot emit light with longer wavelengths, and smaller sized quantum dots emit light with shorter wavelengths. One exemplary nanoparticle useful in hair growth stimulating systems of the present invention includes those produced by Quantum Materials in San Marcos, Texas under the product name ‘Silver Nanoparticles Lipoic Acid Capped’ composed approximately of 0.1% silver by weight and 0.1% (R)-5-(1,2-Dithiolan-3-yl) pentanoic acid by weight.

The nanoparticles (108) of FIG. 1 are dispersed in the topical solution (106) in their natural resting state or, in certain embodiments, the topical solution (106) may be shaken, stirred, or otherwise agitated to disperse the nanoparticles. Whether the nanoparticles (108) of FIG. 1 remain dispersed in the resting state or require agitation to achieve disbursement depends on a variety of factors, including but not limited to the mass of the nanoparticles (108), the viscosity of the topical solution (106), the temperature of the environment, and so on. The nanoparticles (108) of FIG. 1 may also be dispersed by pumping or spraying the topical solution (106) from its container (104) for application to the scalp. Upon application, the topical solution (106), including the nanoparticles (108), is distribute on the scalp (101) among the existing hairs (110).

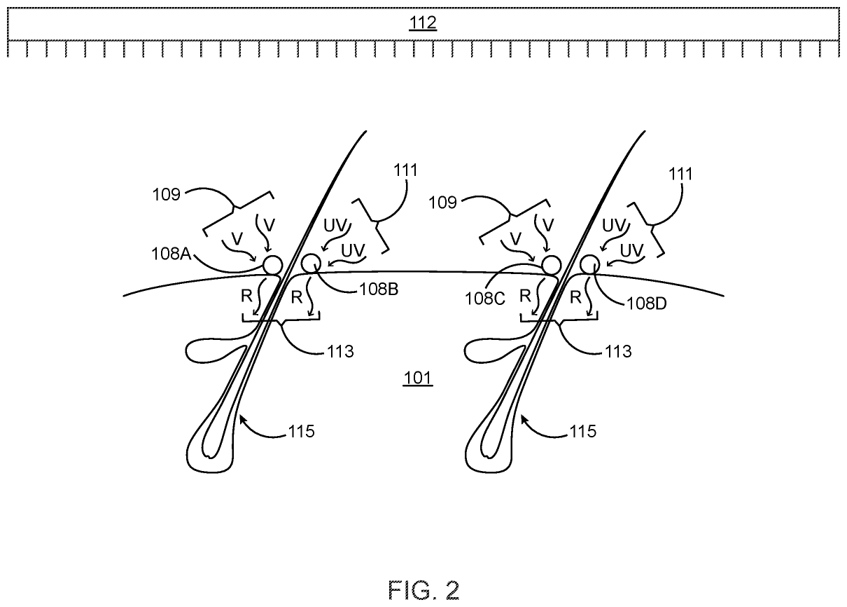

In the example of FIG. 1, the nanoparticles (108) emit red light toward the scalp (101) to stimulate hair growth when irradiated with photons from a light source, which is discussed in more detail with reference to FIG. 2.

Turning to FIG. 2, FIG. 2 sets forth a drawing illustrating a perspective view of the exemplary hair growth stimulating system (100) according to embodiments of the present invention. FIG. 2 is a close up version of the hair growth stimulating system (100) of FIG. 1 after the topical solution (106) along with dispersed nanoparticles (108) are applied to the user's scalp (101). The topical solution (106) applied to the scalp of the user (102) does not appear in FIG. 2, however, because the topical solution (106) has evaporated or has been absorbed into the scalp (101) of the user (102).

The nanoparticles (108) in the example of FIG. 2 are irradiated with photons from a light source (112). A light source useful in embodiments of the present invention could be the sun, overhead lighting such as lighting found in an indoor space, lamps, lighting embedded in a clothing article, or any other source of photons as will occur to those of skill of the art. Types of lighting useful in embodiments of the present invention may include sunlight, incandescent light, florescent light, light-emitting diode light, halogen light, and any other types of lighting as will occur to those of skill in the art. When the nanoparticles (108) of FIG. 2 are irradiated with the photons from a light source, photons striking the nanoparticles (108) result in the excitement of the electrons of the nanoparticles (108) into a higher energy state. In this way, the nanoparticles (108) of FIG. 2 may temporarily store the energy of such photons that collide with the nanoparticles (108) of FIG. 2.

In the example of FIG. 2, photons of various wavelengths strike the nanoparticles (108). Photons (109) with an energy level corresponding to a visible light spectrum irradiate nanoparticles (108A,108C) from the exemplary hair growth stimulating system (100) in FIG. 2. The visible light (V) spectrum falls in the range of the electromagnetic spectrum between ultraviolet light and infrared light and typically ranges in wavelength from approximately 380 nanometers to approximately 750 nanometers. In addition, in the example of FIG. 2, photons (111) with an energy level corresponding to an ultraviolet light spectrum irradiate certain nanoparticles (108B,108D) of the exemplary hair growth stimulating system (100). The ultraviolet light (UV) spectrum falls in the range of the electromagnetic spectrum between visible light and X-rays and typically ranges in wavelength from approximately 10 nanometers to approximately 380 nanometers. As previously mentioned, visible light photons (109) and ultraviolet light photons (111) of FIG. 2 may originate from the sun and reach the nanoparticles on the user's scalp from the user merely being outside in the sunlight. Of course, indoor lighting and other light sources also provide the photons useful in hair growth stimulating systems according to embodiments of the present invention. Although the nanoparticles (108) in Figure are struck by visible light photons (109) and ultraviolet light photons (111), in other embodiments, photons with an energy level corresponding to an infrared light spectrum may irradiate certain nanoparticles useful in the example of FIG. 2 on the scalp of the user (102). The infrared light (IR) spectrum falls in the range of the electromagnetic spectrum between visible light and microwaves and typically ranges in wavelength from approximately 750 nanometers to approximately 1,000,000 nanometers.

In the example of FIG. 2, the nanoparticles (108) may be formed from silver or silver oxide. Other noble metals that are non-toxic to humans may also be used to form exemplary nanoparticles (108). Noble metals are metallic elements that are especially resistant to chemical assault, even at high temperatures. Noble metals display catalytic properties and therefore generally remain unchanged in mass and chemical composition at the end of the reaction.

When the nanoparticles (108) of FIG. 2 are irradiated with photons (109,111) from the light source (112), electrons in the nanoparticles (108) are excited to a state of higher energy. When electrons in the nanoparticles (108) fall down to their previous energy state, those nanoparticles (108) release the excess energy in the form of red light (113) (R) directed toward the scalp (101) to stimulate hair growth. This red light (113) in the example of FIG. 2 consists of photons with an energy level corresponding to the red light spectrum, which generally ranges in wavelength from approximately 620 nanometers to approximately 750 nanometers for visible red light and from approximately 750 nanometers to approximately 1,000,000 nanometers for infrared light. Although, one of skill in the art will recognize that the wavelength boundaries between different portions of the electromagnetic spectrum are approximate values.

Exposing the scalp (101) to red light (113) emitted from the nanoparticles (108) in the example of FIG. 2, allows hair follicles (115) to absorb the red light (113) and hair growth is stimulated. More particularly, the red light (113) of FIG. 2 is absorbed by an intracellular enzyme referred to as ‘Cytochrome C’ and then the enzyme in turn stimulates the follicles (115). Once stimulated, the gene activity in the follicles (115) are promoted. Still further, the red light (113) may increase the level of heat shock proteins (HSPs) such as HSP27 in the follicles (115), which are known to regulate cell growth. The red light (113) of FIG. 2 also has a tendency to increase blood in region of the scalp (101) where the red light (113) penetrates, which also help to stimulate hair growth.

In the example of FIG. 2, the red light (113) emitted from the nanoparticles (108) has a wavelength within the range of 650 to 660 nanometers clustered primarily around the 655 nanometer wavelength. Red light in the 655 nanometer wavelength has demonstrated promising results as it penetrates the scalp and stimulates hair growth. Other wavelengths of red light are also useful in embodiments of the present invention. For example, red light emitted from the nanoparticles of some exemplary hair growth stimulating systems may have a wavelength within the range of 620 to 750 nanometers. In other examples, red light emitted from the nanoparticles of such exemplary hair growth stimulating systems may have a wavelength within the range of 650 to 670 nanometers. Still other embodiments of exemplary hair growth stimulating systems (100) may comprise a plurality of nanoparticles that, when irradiated with photons from a light source, emit red light with a wavelength within the range of 760 to 1,000,000 nanometers.

FIGS. 1 and 2 illustrate embodiments of the present invention in which the topical solution of the exemplary hair growth stimulating system (100) is applied directly to the user's (102) scalp. This configuration provides advantages in that the nanoparticles are deposited directly on the scalp and typically congregate around the openings of scalp where hair follicles exists because of the sloped indentation formed at the opening on the scalp where the hair exits the skin. In this way, red light emitted from such nanoparticles reaches the scalp near the hair follicles without the obstruction of existing hair or other opaque structures that plague traditional laser hair growth systems.

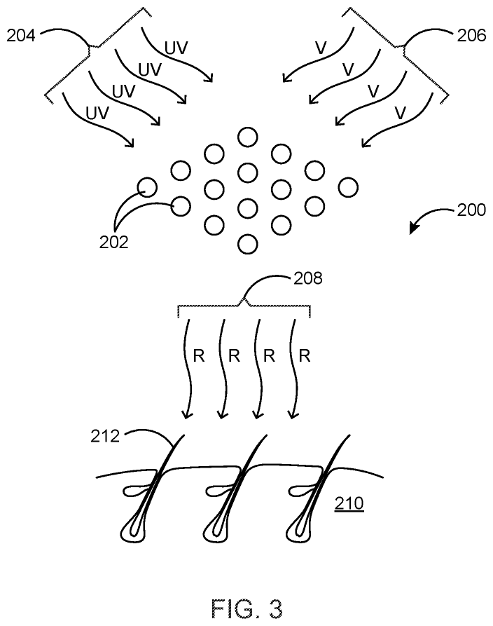

In some embodiments, applying the nanoparticles useful in embodiments of the present invention directly to the scalp may not be ideal. For example, the user may not want to put a topical solution on their scalp because such an application would mess up their existing hair style, the user may not like the feeling of products in their hair or on their scalp, or the user might prefer wearing a head covering on a particular occasion. In such cases, the topical solution with dispersed nanoparticles used in embodiments of the present invention may be applied in a manner differently than described with reference to FIGS. 1 and 2 such that the topical solution with dispersed nanoparticles may be separated by a short distance from a user's scalp, as described with reference to FIG. 3.

FIG. 3 sets forth a drawing illustrating a perspective view of an exemplary hair growth stimulating system (200) according to embodiments of the present invention. The exemplary hair growth stimulating system (200) of FIG. 3 comprises a topical solution with nanoparticles (202) dispersed in the topical solution. The nanoparticles emit red light (208) toward the scalp (210) to stimulate hair grown when irradiated with photons from a light source, such as for example, the sun.

In the example of FIG. 3, photons (206) with an energy level corresponding to the visible (V) light spectrum and photons (204) with an energy level corresponding to the ultraviolet (UV) light spectrum irradiate certain nanoparticles (202) from the exemplary hair growth stimulating system. When irradiated by photons (204, 206), the nanoparticles (202) of FIG. 3 emit a red light (208) toward the scalp (210), which in turn stimulates the growth of hairs (212).

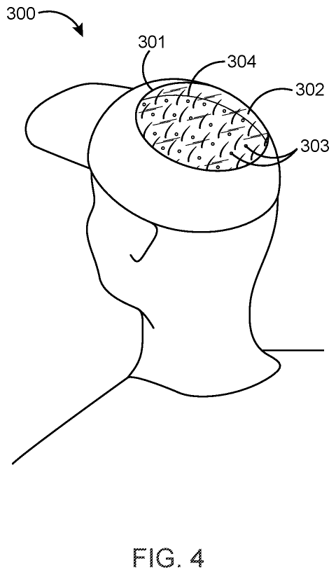

For further explanation of an exemplary hair growth stimulating system in which the nanoparticles are separated from the scalp by a short distance, FIG. 4 sets forth a drawing illustrating a perspective view of an exemplary clothing article for hair growth stimulation according to embodiments of the present invention. The exemplary clothing article of FIG. 4 is a head covering (300) for overlaying on a scalp of a user. The head covering (300) of FIG. 4 has a window (301) to allow light to pass through the head covering (300) to the scalp (304). The window (301) in the example of FIG. 4 has a thin film (302) embedded with nanoparticles (303) that are dispersed throughout the thin film (302). The nanoparticles (303) of FIG. 4 emitting red light toward the scalp to stimulate hair growth when irradiated with photons from a light source.

In the example of FIG. 4, the head covering (300) is implemented as a baseball cap. In other embodiments, a head covering useful in embodiments of the present invention may be implemented as a scarf, kerchief, headband, derby hat, cowboy hat, panama hat, a bowler, Yakama, a ski cap, or any other head covering capable of having window over portions of the scalp to allow light to pass through. Certain embodiments may have a window (301) containing a film made of transparent plastic, while other films may be made of translucent plastic. Other embodiments may contain a window (301) with a film (302) made of lightweight glass or other materials

The nanoparticles (303) of FIG. 4 embedded in the film (302) in the window (301) of the head covering (300) are implemented as quantum dots. When the nanoparticles (303) of FIG. 4, which are embedded in the film (302), are irradiated with photons from a light source, the nanoparticles (303) release energy in the form of red light toward the scalp (304).

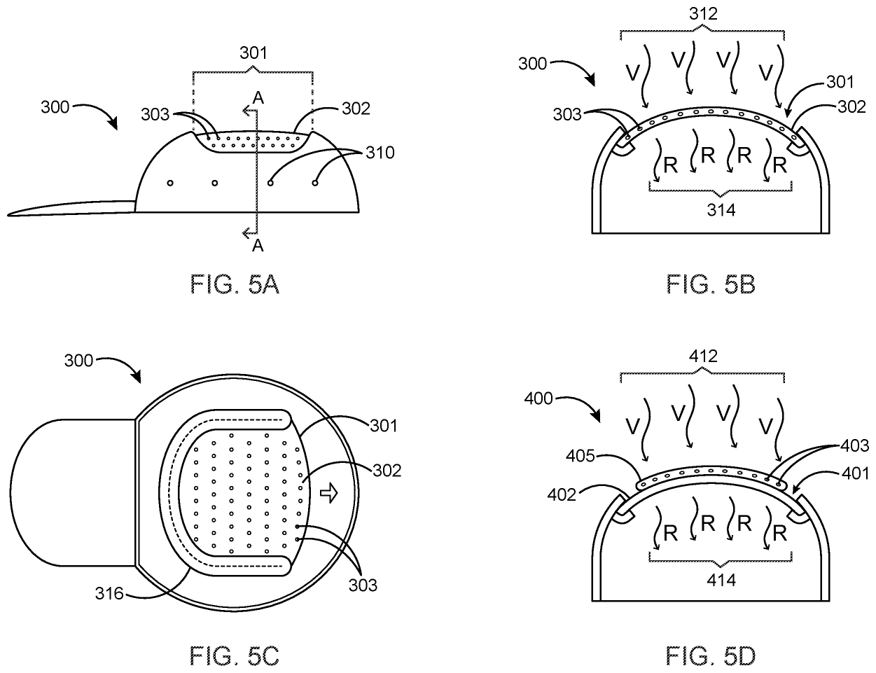

Turning to FIG. 5A, FIG. 5A sets forth a drawing illustrating a right view of an exemplary clothing article of FIG. 4 for hair growth stimulation according to embodiments of the present invention. FIG. 5A depicts a head covering (300) for overlaying a scalp of a user. The head covering (300) of FIG. 5A has a window (301) on the top to allow light to pass through to the scalp. The window has a thin film (302) embedded with nanoparticles (303). The nanoparticles emit red light toward the scalp to stimulate hair growth when irradiated with the photons from a light source such as, for example, the sun. In some exemplary clothing articles useful for hair growth stimulation according to the present invention, the thin film within the window may sit directly on top of the scalp such as, for example, when the clothing article is implemented as a sweat band or Yamaka.

In certain embodiments, the window containing the thin film may be located in other locations or targeted locations on the head covering, other than covering the entire top region, as may be necessary to target the portions of the scalp needing hair growth stimulation.

The head covering (300) of FIG. 5A includes ventilation apertures (310). These ventilation apertures (310) of FIG. 5A provide an exchange of air between the inside and outside of the head covering (300). Such ventilation apertures (310) of FIG. 5A operate to keep the head cooler as the warmer air generating from the user's body heat escapes via the ventilation apertures (310).

FIG. 5B sets forth a drawing illustrating a cross-sectional view along the line A-A of the exemplary clothing article of FIG. 5A for hair growth stimulation according to embodiments of the present invention. If FIG. 5B, the photons (312) having an energy level corresponding to the visible (V) light spectrum irradiate the nanoparticles (303), which in turn emit red light (314) toward the scalp to stimulate hair growth.

FIG. 5C sets forth a drawing illustrating a bottom view of the exemplary clothing article of FIG. 4 for hair growth stimulation according to embodiments of the present invention. The thin film (302), which is embedded with nanoparticles (300), within the window (301) of the head covering (300) of FIG. 5C is held in place between the top of the head covering (300) and the flap (316). The thin film (302) of FIG. 5C may be removed from the head covering (300) by sliding the film (302) down and out of the back of the head covering (300). In the example of FIG. 5C, the film (302) is rigid enough to span the window (301) without sinking in the middle of the file (302) under the film's own weight. Yet, the film is flexible enough to bend in a arc or rounded manner across the window (301) and to bend in a manner to permit easy insertion and removal with respect to the head covering (300).

While the thin film (302) is removed by sliding the film (302) down and out of the back of the head covering (300) in FIG. 5C, in other embodiments, the thin film may be removed by lifting the thin film out of the top of the window of an exemplary head covering. Other embodiments may be comprised of a thin film that may be removed by sliding, snapping, or flipping the thin film out of the window to the side, bottom, front or back of the head covering. In still other embodiments, the thin film may not be removable at all and completely built into the head covering.

In FIGS. 4 and 5A-C, the nanoparticles described with referenced to embodiments of the present invention are embedded in the thin film that is positioned within the window of the exemplary head covering. In certain instances where such films with the embedded nanoparticles are not available but the topical solution with embedded nanoparticles is available, a user may apply the topical solution to directly to the head covering at a specific region so as to still achieve the benefits of red light emission from the nanoparticles to stimulate hair growth. For further explanation, FIG. 5D sets forth a drawing illustrating a cross-sectional view of an exemplary clothing article along a line similar to A-A in FIG. 5A for hair growth stimulation according to embodiments of the present invention.

In FIG. 5D, a head covering (400) for overlaying the scalp of a user has a window (401) to allow light to pass through to the scalp. The window (401) of FIG. 5D has a thin film (402) for receiving a topical solution (405). The topical solution (405) of FIG. 5A includes nanoparticles (403) dispersed throughout the topical solution (405). In the example of FIG. 5A, the nanoparticles (403) emit red light (414) toward the scalp to stimulate hair growth when irradiated with the photons (412) having an energy level corresponding to the visible (V) light spectrum. Of course, as previously mentioned, the photons striking the nanoparticles (403) may also reside in other regions of the electromagnet spectrum including the ultraviolet light and infrared light spectrums.

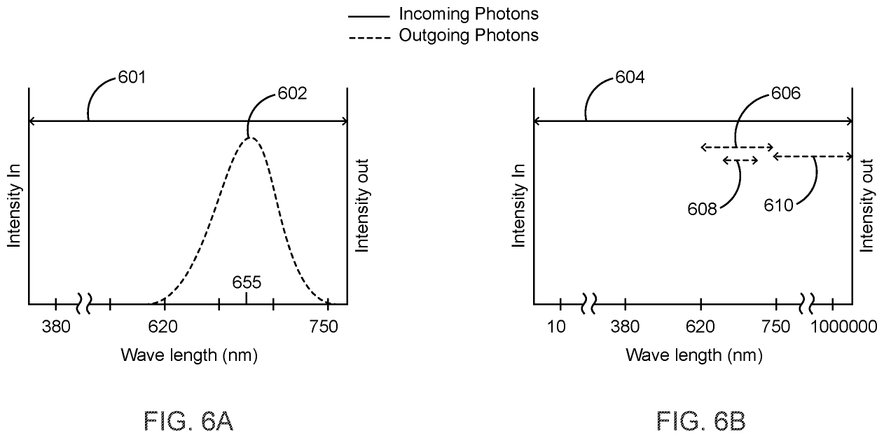

For further explanation regarding the various spectrums of light, FIG. 6A sets forth a drawing illustrating a chart of light wavelengths of incoming photons (601) and outgoing photons (602) of exemplary nanoparticles useful in exemplary clothing articles for hair growth stimulation according to embodiments of the present invention. In FIG. 6A, incoming photons (601) have energy level corresponding to the visible (V) light spectrum, which typically ranges in wavelength from approximately 380 nanometers to approximately 750 nanometers. When those income photons (601) strike the nanoparticles, the nanoparticles absorb the energy of the incoming photon (601) as electrons of the nanoparticles move to a higher energy state—only to drop out of that higher energy state immediately thereafter. As the electrons drop out of that higher energy state, the nanoparticles release outgoing photons (602)—typically from the opposite side of the nanoparticles as where the incoming photons (601) struck the nanoparticles. In FIG. 6A, the outgoing photons (602) have an energy level corresponding to the red light spectrum, which generally ranges in wavelength from approximately 620 nanometers to approximately 750 nanometers, with most of the outgoing photons having a wavelength around 655 nanometers, which has been shown to facilitate hair growth.

FIG. 6B sets forth a drawing illustrating a chart of light wavelengths of incoming photons (604) and outgoing photons (606, 608, 610) of exemplary nanoparticles useful in exemplary clothing articles for hair growth stimulation according to embodiments of the present invention. In FIG. 6B, incoming photons (604) have energy level corresponding to the ultraviolet (UV), visible (V), and infrared (IR) light spectrums, which all together typically range in wavelength from approximately 10 nanometers to approximately 1,000,000 nanometers. In FIG. 6B, the outgoing photons (606) have an energy level corresponding to the red light spectrum, which generally ranges in wavelength from approximately 620 nanometers to approximately 750 nanometers, as discussed with reference to FIG. 6A. The outgoing photons (608) of FIG. 6B, however, have an energy level corresponding to a narrower range within the red light spectrum. The narrow range of outgoing photons (608) of FIG. 6B consists of photons having energy levels that correspond with wavelengths between 650 nanometers and 670 nanometers. An even narrower range that might prove useful in embodiments of the present invention may include photons having energy levels that correspond with wavelengths between 650 nanometers and 660 nanometers, which should provide more red light centered around the 655 nanometer wavelength that has been shown to stimulate hair growth. In the example of FIG. 6B, the outgoing photons (610) have an energy level corresponding to the infrared light spectrum, which generally ranges in wavelength from approximately 760 nanometers to approximately 1,000,000 nanometers.

Readers will immediately recognize the benefits of the exemplary hair growth stimulating systems and exemplary clothing articles for hair growth stimulation described with reference to the accompany drawings. By promoting the growth of a user's own hair using quantum dots, users avoid the physical limitations of mechanical solutions such as wigs and toupees, avoid the expense and hassle of obtaining prescriptions for pharmaceutical grade hair grow stimulators that do not provide the targeted benefits of the present invention, avoid skin burns, irritations, and uneven application associated with lasers, avoid the negative affects associated with using medicated shampoos, and forgo costly and painful surgical remedies. Exemplary hair growth stimulating systems and exemplary clothing articles for hair growth stimulation in according to embodiments of the present invention provide a superior treatment of hair loss because such systems and clothing articles can be utilized at home by a person and worn all day without worrying about damaging the skin due to prolonged exposure.

While certain exemplary embodiments have been described in detail and shown in the accompanying drawings, it is to be understood that such embodiments are merely illustrative of and not devised without departing from the basic scope thereof, which is determined by the claims that follow.

What I claim is:

1. A hair growth stimulating system comprising:
a topical solution for application on a scalp of a user; and
a plurality of nanoparticles dispersed in the topical solution, the nanoparticles emitting red light toward the scalp to stimulate hair growth when irradiated with photons from a light source.

2. The system of claim 1 wherein the red light has a wavelength within the range of 620 to 750 nanometers.

3. The system of claim 1 wherein the red light has a wavelength within the range of 650 to 670 nanometers.

4. The system of claim 1 wherein the red light has a wavelength within the range of 650 to 660 nanometers.

5. The system of claim 1 wherein the red light has a wavelength within the range of 760 to 1,000,000 nanometers.

6. The system of claim 1 wherein the photons have an energy level corresponding to a visible light spectrum.

7. The system of claim 1 wherein the photons have an energy level corresponding to an ultraviolet light spectrum.

8. The system of claim 1 wherein the photons have an energy level corresponding to an infrared light spectrum.

9. The system of claim 1 wherein the topical solution includes alpha lipoic acid.

10. The system of claim 1 wherein the topical solution includes glutathione.

11. The system of claim 1 wherein the nanoparticles are formed from silver.

12. The system of claim 1 wherein the nanoparticles are formed from a noble metal.

13. A clothing article for hair growth stimulation comprising:
a head covering for overlaying a scalp of a user, the head covering having a window to allow light to pass through to the scalp, the window having a thin film embedded with nanoparticles, the nanoparticles emitting red light toward the scalp to stimulate hair growth when irradiated with photons from a light source.

14. The clothing article of claim 13 wherein the red light has a wavelength within the range of 650 to 660 nanometers.

15. The clothing article of claim 13 wherein the head covering is a cap.

16. The clothing article of claim 13 wherein the thin film is removeable.

17. A clothing article for hair growth stimulation comprising:
a head covering for overlaying a scalp of a user, the head covering having a window to allow light to pass through to the scalp, the window having a thin film for receiving a topical solution, the topical solution including nanoparticles dispersed in the topical solution, the nanoparticles emitting red light toward the scalp to stimulate hair growth when irradiated with photons from a light source.

18. The clothing article of claim 17 wherein the red light has a wavelength within the range of 650 to 660 nanometers.

19. The clothing article of claim 17 wherein the head covering is a cap.

20. The clothing article of claim 17 wherein the thin film is removeable.
